(19) United States
(12) Patent Application Publication
(10) Pub. No.: US 2024/0051914 A1
(43) Pub. Date: Feb. 15, 2024

(54) THE HEXADECANE TROMETHAMINE COMPOUND, ITS SYNTHESIS METHOD AND ITS APPLICATION IN ANTITUMOR AND ANTIFUNGAL ASPECTS

(71) Applicant: Wuxi No.2 People's Hospital, Wuxi City (CN)

(72) Inventors: Zejun Pei, Wuxi City (CN); Xin Sun, Wuxi City (CN); Jingyu Zhu, Wuxi City (CN); Xin Wang, Wuxi City (CN); Xin Yang, Wuxi City (CN); Renjing Hu, Wuxi City (CN); Yi Qian, Wuxi City (CN)

(21) Appl. No.: 17/998,119

(22) PCT Filed: Mar. 18, 2022
(86) PCT No.: PCT/CN2022/081648

§ 371 Date: Nov. 7, 2022

(30) Foreign Application Priority Data
Apr. 25, 2021 (CN) 202110448347.2

(51) Int. Cl. C07C 215/10; A61K 31/133; C07C 213/10; C07C 213/08; A61P 35/00; A61P 31/10

(52) U.S. Cl. CPC C07C 215/10; A61K 31/133; C07C 213/10; C07C 213/08; A61P 35/00; A61P 31/10

(57) ABSTRACT

The invention belongs to the field of organic synthesis, and specifically relates to the hexadecane tromethamine compound which has the following structure:

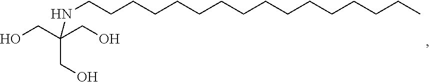 [SMILES: CCCCCCCCCCCCCCCCNC(CO)(CO)CO]

and provides the synthesis method of the hexadecane tromethamine compound, and the application of the hexadecane tromethamine compound in anti-tumor and antifungal aspects. The invention fills the gaps in the synthesis process of the hexadecane tromethamine compound and the application of the hexadecane tromethamine compound in anti-tumor and antifungal aspects.

### FIELD OF TECHNOLOGY

The invention belongs to the field of organic synthesis, and specifically relates to the hexadecane tromethamine compound, the synthesis method of the hexadecane tromethamine compound, and the application of the hexadecane tromethamine compound in anti-tumor and antifungal aspects.

### BACKGROUND

In recent years, the morbidity and mortality of cancer our country remains stubbornly high, still a serious disease that threatens human health. The rising rate of tumor morbidity has also made significant growth in antineoplastic drugs market. The average growth rate of the antineoplastic drugs market has exceeded 15% in the past five years, which is significantly higher than the growth of the overall medical market. In the top ten therapeutic field of new medicine special support, the anti-tumor drugs take up the biggest share. In order to improve the therapy effects for malignant tumor, the research of innovative medicines need to be increased. Although anti-tumor drug will account for about one-third in the expected new drug reaching the market, due to the specificity of tumor treatment, including recurrence, drug resistance, etc., there is still a need to strengthen the efforts to develop various anti-cancer mechanisms for meet the individual demand of treating tumor.

In contrast, the development of antifungal drugs is relatively slow. Fungal infection is one of the main infectious diseases in clinical, which is classified as superficial fungal infection and invasive fungal infection. Thereinto, the morbidity and case fatality rate of invasive fungal diseases have increased year by year in recent decades. At present, there is not much optional medicinal species of treating fungal infections, mainly polyene drugs, pyrrole drugs, echinocandins drugs and 5-flucytosine (5-Fc) drugs. The polylene and pyrrole antifungal drugs often have an element of liver and kidney toxicity and other adverse reactions. It is easy for 5-flucytosine drugs to occur fungal resistance, so 5-flucytosine drugs are not generally used alone. The echinocandins drugs, the representative drugs of which are caspofungin, micafungin, etc, are relatively new and strong antifungal. Due to the insufficiency of the optional species and quantity of clinical antifungal drugs, the antifungal resistance is also becoming increasingly serious. Even much “super fungi” occurring repeatedly have become resistant to caspofungin, micafungin, etc which are the last line of defense to against fungi, which seriously threaten the health and safety of patients. Therefore, for the current science and technology workers, it is very urgent to solve the problem of finding more and better new antifungal drugs as soon as possible, to overcome the problem of antifungal resistance. In summary, anti-tumor and antifungal drugs are the hot point and forward position fields in developing the new pharmaceutical.

### Technical Problem

In order to overcome the shortcomings of existing technology, the present invention provides the hexadecane tromethamine compound which fills the gaps in the hexadecane tromethamine, the synthesis process of the hexadecane tromethamine compound and the application of the hexadecane tromethamine compound in anti-tumor and antifungal aspects.

### Technical Solution

In order to achieve the above purpose, the solution of the invention is that:

The hexadecane tromethamine compound is a hexadecyl tromethamine or a hexadecyl trometamol salt, and the compound has the following structure:

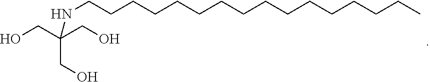 [SMILES: CCCCCCCCCCCCCCCCNC(CO)(CO)CO]

The synthesis method of the hexadecane tromethamine was synthesized by oil bath reflux with ttrihydroxymethyl aminomethane and n-hexadecyl bromide.

The detailed procedures of the synthesis method comprises the steps of: step 1, dissolving trihydroxymethyl aminomethane and n-hexadecyl bromide in anhydrous ethanol, and stirring until evenly dispersed; step 2, adding sodium carbonate into the solution of step 1, and stirring by oil bath reflux for 20 h and then cooling to room temperature, and adding water and stirring, and the crude product was obtained after filtration.

The temperature of the oil bath is 80° C.

The synthesis method also comprises step 3, purifying the crude product.

The purification of the crude product is by means of washing by methyl tert-butyl ether and hydrochloric acid, and the white solid which is 2-(hexadecyl amino)-2-(hydroxymethyl) propane-1,3-diol hydrochloride was obtained after filtration.

The concentration of the hydrochloric acid was 1 M.

The hexadecyl trometamol salt was synthesized by the reaction of the hexadecyl tromethamine with acid.

The application of the hexadecane tromethamine compound is in preparing anti-tumor drugs.

The application of the hexadecane tromethamine compound is in preparing antifungal drugs.

### BENEFICIAL EFFECTS

1. The present invention fills the gaps in the hexadecyl tromethamine and its salts, and also fills the gaps in the technology of the hexadecane tromethamine compound.
2. The present invention uses bromide and methylamine to form a reaction in anhydrous ethanol system, and forms a reflux system with the action of sodium carbonate to realize a long chain substitution of tromethamine.
3. The hexadecyl tromethamine provided by the present invention has a strong anti-tumor and antifungal biological activity.
4. The hexadecyl tromethamine provided by the present invention can be used in antifungal fields and anti-tumor fields.

### DESCRIPTION OF THE DRAWINGS

FIG. 1 depicts a Hydrogen Nuclear Magnetic Resonance of the hexadecyl tromethamine hydrochloride salt in the embodiment of present invention;

FIG. 2 depicts an ESI-MS analysis diagram of the hexadecyl tromethamine hydrochloride salt.

FIG. 3 depicts an inhibition curve of the hexadecyl tromethamine on the gastric cancer cell HGC-27.

FIG. 4 depicts a Hydrogen Nuclear Magnetic Resonance of the hexadecyl tromethamine in the embodiment of present invention.

### DETAIL DESCRIPTION

An embodiment of the present invention will now be described by referring to the accompanying drawings which will be not any restriction for the claims of this invention.

### Example 1

The Hexadecyl Tromethamine Hydrochloride Salt

Synthetic Method:

5 g of trihydroxymethyl aminomethane (41.3 mmol) and 10 g of n-hexadecyl bromide were dissolved in 33 mL of ethanol, and then 7.0 g of sodium carbonate (66 mmol) was added. Then the temperature of oil bath had risen to 80° C., and the solution was stirred with reflux for 20 h. Then the solution was cooled to room temperature, and added with 170 mL of water, and stirred well. The crude product of the hexadecyl tromethamine was obtained after filtration.

The crude product of the hexadecyl tromethamine was added into the methyl tert-butyl ether and 1 M HCL to be washed, and the white solid which is 2-(hexadecyl amino)-2-(hydroxymethyl) propane-1,3-diol hydrochloride was obtained after filtration. The mass of the white solid was 6.6 g, and the yield of the white solid was 57%.

The hexadecyl tromethamine hydrochloride salt has the following structure:

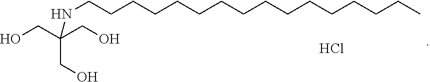 [SMILES: CCCCCCCCCCCCCCCCNC(CO)(CO)CO ; Cl]

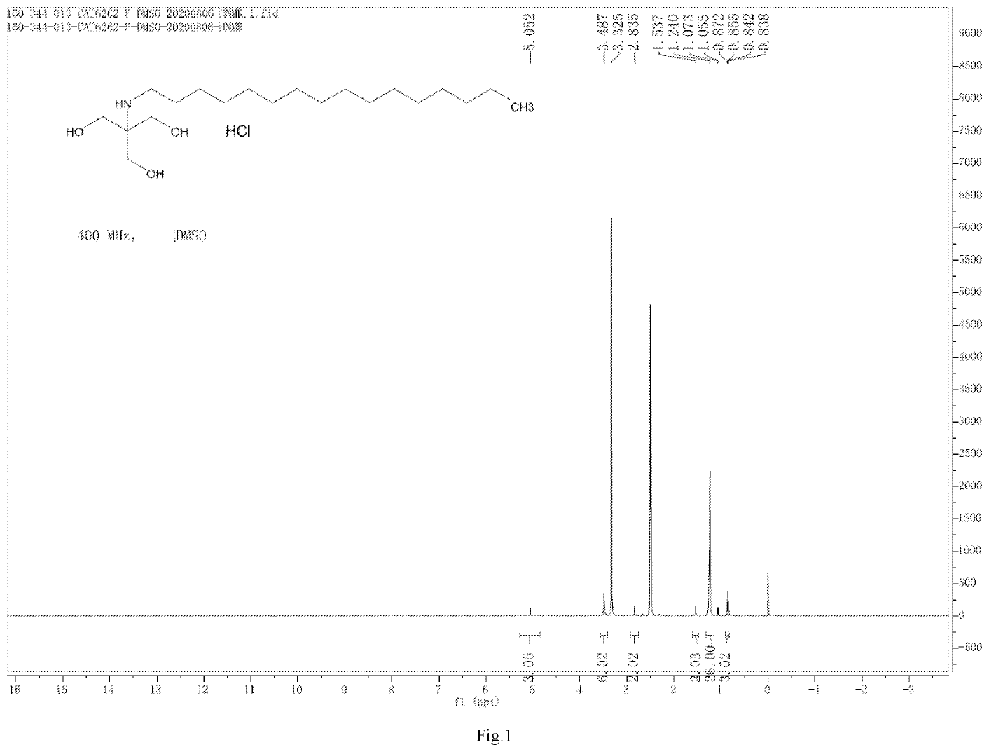

FIG. 1 is the result of Hydrogen Nuclear Magnetic Resonance of the product. According to the analysis of HMNR, position 0.838 should be three hydrogen ions on the —CH3; position 1-1.5 should be 28 hydrogen ions on the straight-chain of the hexadecyl; position 3.325-3.487 should be the hydrogen ion on the —CH2- connecting the hydroxyl; position 5.05 should be the hydrogen ion on the hydroxyl. From the above hydrogen spectrum analysis, the distribution of hydrogen ions is the same as the hydrogen ion distribution of the hexadecyl tromethamine.

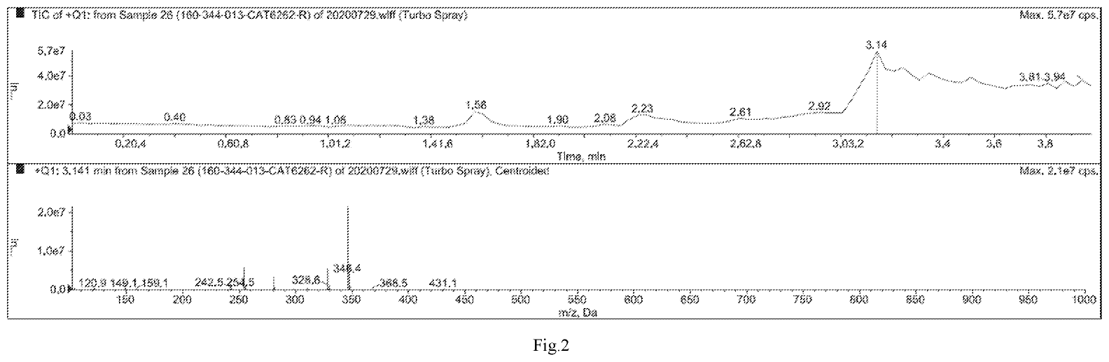

FIG. 2 is the analysis result of ESI-MS. Judging from ion fragments, it can also be determined that the product is the hexadecyl tromethamine.

Performance Detection

1. The Antitumor Function Detection of the Hexadecyl Tromethamine Hydrochloride Salt

Test Method: The logarithmic phase cells were seeded in 96-well plates with 3,000 cells per well/100 μl of density. After the cells were adhered, 100 μl of the test compounds with different concentrations were added. Design 6-8 concentration gradients. Set five parallel wells in per group and set the control group. After the compound and tumor cells were incubated for 72 hours, 10 μl of CCK-8 solution was added in per well. After the cells were incubated for 1-2 hours in the incubator, the optical density (OD) of per well was counted by Universal Microplate Spectrophotometer to calculate the inhibition ratio: inhibition ratio (IR %)=(1−TOD/COD)×100%, TOD: the mean OD of drug administration group, COD: the mean OD of solvent control. The dose-response curve was plotted in accordance to the drugs with different concentrations and the inhibition ratio of cells to obtain the Inhibitory concentration of drug (IC₅₀).

The detection results are as follows:

| | |
|---|---|
| | The inhibition ratio of the hexadecyl |
| Carcinoma | tromethamine hydrochloride salt (%) |

| cell lines | 20 μM | 2 μM |
|---|---|---|
| MKN-45 | 99% | 31% |
| HGC-27 | 101% | 72% |
| SGC-7901 | 100% | 21% |
| AGS | 99% | 42% |

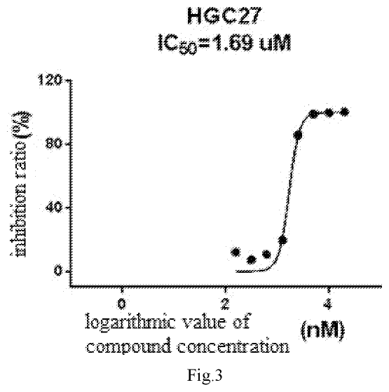

Thereinto, the inhibition of the hexadecyl tromethamine hydrochloride salt on the gastric cancer cell HGC-27 was as shown in FIG. 3. When the logarithmic value of compound concentration reached 3 nM, the inhibitory effect rose rapidly until it reaches 100%.

According to FIG. 3 and the above table, the hexadecyl tromethamine hydrochloride salt can exert a strong anti-proliferation effect with reaching a certain concentration.

2. The Anti-Fungal Function Detection of the Hexadecyl Tromethamine Hydrochloride Salt

Test Method: The hexadecyl tromethamine hydrochloride salt solution was semi-diluted to five concentration gradients (100 μg/ml, 50 μg/ml, 25 μg/ml, 12.5 μg/ml and 6.25 μg/ml) by RPMI1640 fluid nutrient medium. 100 μl of every gradient solution was added in the 96-well plate for standby application. Standard fungal strain and clinical drug-resistant fungal strain in table 2 were chosen as the experimental fungus. The activation of the experimental fungus was obtained through that the experimental fungus were cultivated for 48 h at 30° C. The activated experimental fungus was matched to fungal suspension. Count and adjust the concentration using blood counting chamber. The moderate fungal suspension were added into RPMI1640 fluid nutrient medium to 0.5-2.5×10³ cfμ/ml of final working concentration. 100 μl of above fungal suspension was added into the above 96-well plate with the hexadecyl tromethamine hydrochloride salt solution. Two parallel wells were set for each concentration gradient compound of each strain. Blank medium and blank medium with fungal solution were set as control simultaneously. All 96-well plates were placed in incubator and incubated for 35 and 24 h, and the experimental results were shown in the table below:

| | The hexadecyl tromethamine hydrochloride salt | |
|---|---|---|
| Category | (Minimum Inhibitory Concentration, MIC) | |
| Clinical drug-resistant | Candida tropicalis | 25 μg/ml |
| fungal strain | 191529 | |
| | Candida tropicalis | 25 μg/ml |
| | 191327 | |
| | Candida parapsilosis | 25 μg/ml |
| | 191344 | |
| Standard fungal strain | Candida glabrata | 25 μg/ml |
| | 337348 | |
| | Candida krusei | 50 μg/ml |
| | 185429 | |
| | Candida lusitaniae | 25 μg/ml |
| | 340928 | |

These data indicate that the hexadecyl tromethamine hydrochloride salt has a good antifungal effect.

### Example 2

The Hexadecyl Tromethamine

Synthetic Method:

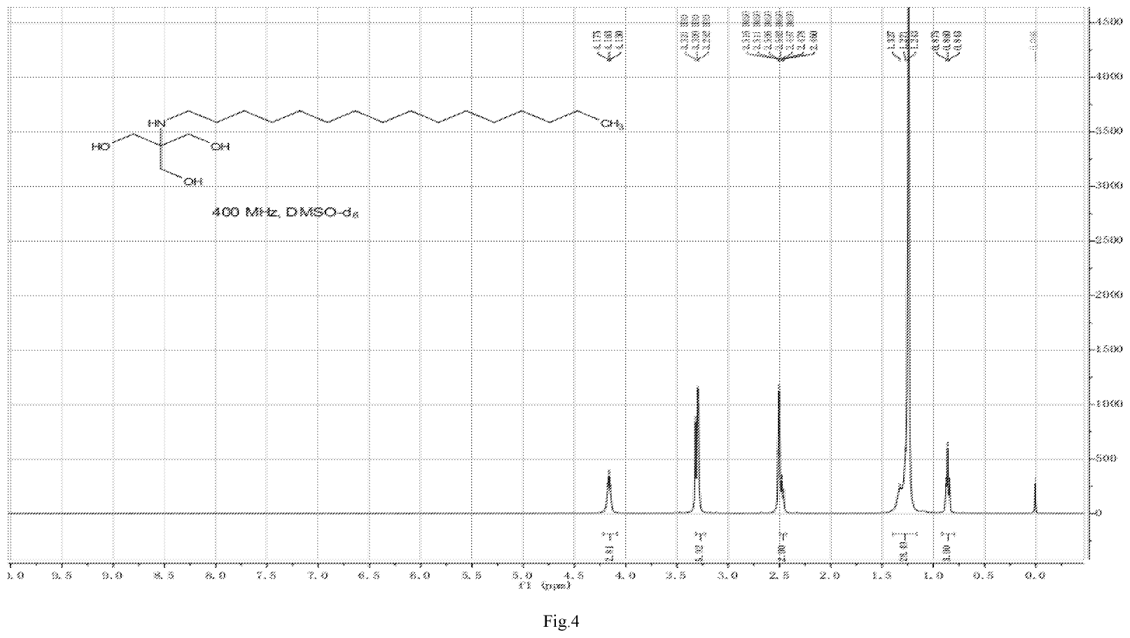

5 g of trihydroxymethyl aminomethane (41.3 mmol) and 10 g of n-hexadecyl bromide were dissolved in 33 mL of ethanol, and then 7.0 g of sodium carbonate (66 mmol) was added. Then the temperature of oil bath had risen to 80° C., and the solution was stirred with reflux for 20 h. Then the solution was cooled to room temperature, and added with 170 mL of water, and stirred well. The crude product of the hexadecyl tromethamine was obtained after filtration. The crude product of the hexadecyl tromethamine was added into the methyl tert-butyl ether and 1 M HCL to be washed, and the white solid which is 2-(hexadecyl amino)-2-(hydroxymethyl) propane-1,3-diol hydrochloride was obtained after filtration. The hexadecyl tromethamine hydrochloride salt is dissolved with water, then the sodium bicarbonate solution was added to alkalinize, recrystallize, filter and wash. The pure hexadecyl tromethamine was obtained. The result of Hydrogen Nuclear Magnetic Resonance of the product was shown in FIG. 4.

The hexadecyl tromethamine has the following structure:

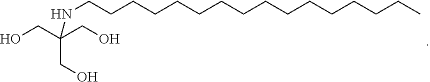 [SMILES: CCCCCCCCCCCCCCCCNC(CO)(CO)CO]

1. The Antitumor Function Detection of the Hexadecyl Tromethamine

Test Method: The logarithmic phase cells were seeded in 96-well plates with 3,000 cells per well/100 l of density. After the cells were adhered, 100 l of the test compounds with different concentrations were added. Design 6-8 concentration gradients. Set five parallel wells in each group and set the control group. After the compound and tumor cells were incubated for 72 hours, 10 l of CCK-8 solution was added in each well. After the cells were incubated for 1-2 hours in the incubator, the optical density (OD) of per well was counted by Universal Microplate Spectrophotometer to calculate the inhibition ratio: inhibition ratio (IR %)=(1−TOD/COD)×100%, TOD: the mean OD of drug administration group, COD: the mean OD of solvent control. The dose-response curve was plotted in accordance to the drugs with different concentrations and the inhibition ratio of cells to obtain the Inhibitory concentration of drug (IC₅₀).

The detection results are as follows:

| | |
|---|---|
| | The inhibition ratio of the |
| Carcinoma | hexadecyl tromethamine (%) |

| cell lines | 20 μM | 2 μM |
|---|---|---|
| MKN-45 | 98% | 32% |
| HGC-27 | 100% | 72% |
| SGC-7901 | 102% | 24% |
| AGS | 99% | 43% |

2. The Anti-Fungal Function Detection of the Hexadecyl Tromethamine

Test Method: The hexadecyl tromethamine solution was semi-diluted to five concentration gradients (100 μg/ml, 50 μg/ml, 25 μg/ml, 12.5 μg/ml and 6.25 μg/ml) by RPMI1640 fluid nutrient medium. 100 μl of every gradient solution was added in the 96-well plate for standby application. Standard fungal strain and clinical drug-resistant fungal strain in table 2 were chosen as the experimental fungus. The activation of the experimental fungus was obtained through that the experimental fungus were cultivated for 48 h at 30° C. The activated experimental fungus was matched to fungal suspension. Count and adjust the concentration using blood counting chamber. The moderate fungal suspension were added into RPMI1640 fluid nutrient medium to 0.5-2.5×10³ cfμ/ml of final working concentration. 100 μl of above fungal suspension was added into the above 96-well plate with the hexadecyl tromethamine solution. Two parallel wells were set for each concentration gradient compound of each strain. Blank medium and blank medium with fungal solution were set as control simultaneously. All 96-well plates were placed in incubator and incubated for 35 and 24 h, and the experimental results were shown in the table below:

| | The hexadecyl tromethamine | |
|---|---|---|
| Category | (Minimum Inhibitory Concentration, MIC) | |
| Clinical drug-resistant | Candida tropicalis | 25 μg/ml |
| fungal strain | 191529 | |
| | Candida tropicalis | 25 μg/ml |
| | 191327 | |
| | Candida parapsilosis | 25 μg/ml |
| | 191344 | |
| Standard fungal strain | Candida glabrata | 25 μg/ml |
| | 337348 | |
| | Candida krusei | 50 μg/ml |
| | 185429 | |
| | Candida lusitaniae | 25 μg/ml |
| | 340928 | |

### Example 3

The Hexadecyl Tromethamine Phosphate

Synthetic Method:

1.5 g of pure hexadecyl tromethamine was mixed with 0.5 g of 10 mol/L phosphate. After sufficient reaction, the solvent was removed and the residue was recrystallized with absolute ethanol to obtain the white solid. The mass of the white solid was 1.2 g, and the yield of the white solid was 62%.

The hexadecyl tromethamine phosphate has the following structure:

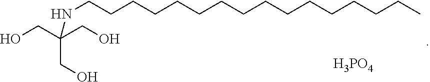

1. The Antitumor Function Detection of the Hexadecyl Tromethamine Phosphate

Test Method: The logarithmic phase cells were seeded in 96-well plates with 3,000 cells per well/100 μl of density. After the cells were adhered, 100 μl of the test compounds with different concentrations were added. Design 6-8 concentration gradients. Set five parallel wells in each group and set the control group. After the compound and tumor cells were incubated for 72 hours, 10 l of CCK-8 solution was added in each well. After the cells were incubated for 1-2 hours in the incubator, the optical density (OD) of per well was counted by Universal Microplate Spectrophotometer to calculate the inhibition ratio: inhibition ratio (IR %)=(1−TOD/COD)×100%, TOD: the mean OD of drug administration group, COD: the mean OD of solvent control. The dose-response curve was plotted in accordance to the drugs with different concentrations and the inhibition ratio of cells to obtain the Inhibitory concentration of drug (IC₅₀).

The detection results are as follows:

| | |
|---|---|
| | The inhibition ratio of the hexadecyl |
| Carcinoma | tromethamine phosphate (%) |

| cell lines | 20 μM | 2 μM |
|---|---|---|
| MKN-45 | 97% | 29% |
| HGC-27 | 99% | 68% |
| SGC-7901 | 101% | 25% |
| AGS | 100% | 44% |

2. The Anti-Fungal Function Detection of the Hexadecyl Tromethamine Phosphate

Test Method: The hexadecyl tromethamine phosphate solution was semi-diluted to five concentration gradients (100 μg/ml, 50 μg/ml, 25 μg/ml, 12.5 μg/ml and 6.25 g/ml) by RPMI1640 fluid nutrient medium. 100 μl of every gradient solution was added in the 96-well plate for standby application. Standard fungal strain and clinical drug-resistant fungal strain in table 2 were chosen as the experimental fungus. The activation of the experimental fungus was obtained through that the experimental fungus were cultivated for 48 h at 30° C. The activated experimental fungus was matched to fungal suspension. Count and adjust the concentration using blood counting chamber. The moderate fungal suspension were added into RPMI1640 fluid nutrient medium to 0.5-2.5×10³ cfμ/ml of final working concentration. 100 μl of above fungal suspension was added into the above 96-well plate with the hexadecyl tromethamine phosphate solution. Two parallel wells were set for each concentration gradient compound of each strain. Blank medium and blank medium with fungal solution were set as control simultaneously. All 96-well plates were placed in incubator and incubated for 35 and 24 h, and the experimental results were shown in the table below:

| | The hexadecyl tromethamine phosphate | |
|---|---|---|
| Category | (Minimum Inhibitory Concentration, MIC) | |
| Clinical drug-resistant | Candida tropicalis | 25 μg/ml |
| fungal strain | 191529 | |
| | Candida tropicalis | 25 μg/ml |
| | 191327 | |
| | Candida parapsilosis | 50 μg/ml |
| | 191344 | |
| Standard fungal strain | Candida glabrata | 50 μg/ml |
| | 337348 | |
| | Candida krusei | 50 μg/ml |
| | 185429 | |
| | Candida lusitaniae | 25 μg/ml |
| | 340928 | |

These data indicate that the hexadecyl tromethamine phosphate has a good antifungal effect.

### Example 4

The Hexadecyl Tromethamine Benzene Sulfonate

Synthetic Method:

1.5 g of pure hexadecyl tromethamine was dissolved in 80 mL of ethanol, and 0.85 g of benzene sulfonic acid monohydrate was added under stirring at room temperature, heated to reflux for 1 h. Cool, and the solid was precipitated to be filtered. The white solid was obtained after full drying. After sufficient reaction, the solvent was removed and the residue was recrystallized with absolute ethanol to obtain the white solid. The mass of the white solid was 2.1 g, and the yield of the white solid was 92%.

The hexadecyl tromethamine benzene sulfonate has the following structure:

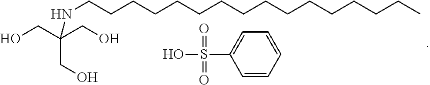

1. The Antitumor Function Detection of the Hexadecyl Tromethamine Benzene Sulfonate

Test Method: The logarithmic phase cells were seeded in 96-well plates with 3,000 cells per well/100 μl of density. After the cells were adhered, 100 μl of the test compounds with different concentrations were added. Design 6-8 concentration gradients. Set five parallel wells in each group and set the control group. After the compound and tumor cells were incubated for 72 hours, 10 l of CCK-8 solution was added in each well. After the cells were incubated for 1-2 hours in the incubator, the optical density (OD) of per well was counted by Universal Microplate Spectrophotometer to calculate the inhibition ratio: inhibition ratio (IR %)=(1−TOD/COD)×100%, TOD: the mean OD of drug administration group, COD: the mean OD of solvent control. The dose-response curve was plotted in accordance to the drugs with different concentrations and the inhibition ratio of cells to obtain the Inhibitory concentration of drug (IC₅₀).

The detection results are as follows:

| | |
|---|---|
| | The inhibition ratio of the hexadecyl |
| Carcinoma | tromethamine benzene sulfonate (%) |

| cell lines | 20 μM | 2 μM |
|---|---|---|
| MKN-45 | 100% | 34% |
| HGC-27 | 96% | 62% |
| SGC-7901 | 98% | 23% |
| AGS | 95% | 38% |

2. The Anti-Fungal Function Detection of the Hexadecyl Tromethamine Benzene Sulfonate Test Method: The hexadecyl tromethamine benzene sulfonate solution was semi-diluted to five concentration gradients (100 μg/ml, 50 μg/ml, 25 μg/ml, 12.5 μg/ml and 6.25 g/ml) by RPMI1640 fluid nutrient medium. 100 μl of every gradient solution was added in the 96-well plate for standby application. Standard fungal strain and clinical drug-resistant fungal strain in table 2 were chosen as the experimental fungus. The activation of the experimental fungus was obtained through that the experimental fungus were cultivated for 48 h at 30° C. The activated experimental fungus was matched to fungal suspension. Count and adjust the concentration using blood counting chamber. The moderate fungal suspension were added into RPMI1640 fluid nutrient medium to 0.5-2.5×10³ cfμ/ml of final working concentration. 100 μl of above fungal suspension was added into the above 96-well plate with the hexadecyl tromethamine benzene sulfonate solution. Two parallel wells were set for each concentration gradient compound of each strain. Blank medium and blank medium with fungal solution were set as control simultaneously. All 96-well plates were placed in incubator and incubated for 35 and 24 h, and the experimental results were shown in the table below:

| | The hexadecyl tromethamine benzene sulfonate | |
|---|---|---|
| Category | (Minimum Inhibitory Concentration, MIC) | |
| Clinical drug-resistant | Candida tropicalis | 50 μg/ml |
| fungal strain | 191529 | |
| | Candida tropicalis | 50 μg/ml |
| | 191327 | |
| | Candida parapsilosis | 50 μg/ml |
| | 191344 | |
| Standard fungal strain | Candida glabrata | 25 μg/ml |
| | 337348 | |
| | Candida krusei | 100 μg/ml |
| | 185429 | |
| | Candida lusitaniae | 25 μg/ml |
| | 340928 | |

These data indicate that the hexadecyl tromethamine benzene sulfonate has a good antifungal effect.

It is understandable that the above specific description of the invention is used only to describe the invention and is not limited by the technical solution described in the embodiments of the invention. The ordinary skilled artisan in the field should understand that the invention can still be modified or equivalently replaced to achieve the same technical effect; Various changes and modifications may be within the scope of protection of the present invention, as long as they serve the demand of use.

1. The hexadecane tromethamine compound, which is characterized in that the compound is a hexadecyl tromethamine or a hexadecyl trometamol salt, and the compound has the following structure:

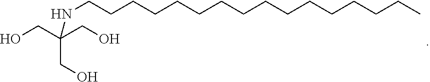 [SMILES: CCCCCCCCCCCCCCCCNC(CO)(CO)CO]

2. The hexadecane tromethamine compound according to claim 1, which is characterized in that the synthesis method of the hexadecane tromethamine was synthesized by oil bath reflux with trihydroxymethyl aminomethane and n-hexadecyl bromide.

3. The hexadecane tromethamine compound according to claim 2, which is characterized in that the detailed procedures of the synthesis method comprise the steps of: step 1, dissolving trihydroxymethyl aminomethane and n-hexadecyl bromide in anhydrous ethanol, and stirring until evenly dispersed; step 2, adding sodium carbonate into the solution of step 1, and stirring by oil bath reflux for 20 h and then cooling to room temperature, and adding water and stirring, and the crude product was obtained after filtration.

4. The hexadecane tromethamine compound according to claim 3, which is characterized in that the temperature of the oil bath is 80° C.

5. The hexadecane tromethamine compound according to claim 3, which is characterized in that the synthesis method also comprises step 3, purifying the crude product.

6. The hexadecane tromethamine compound according to claim 5, which is characterized in that the purification of the crude product is by means of washing by methyl tert-butyl ether and hydrochloric acid, and the white solid which is 2-(hexadecyl amino)-2-(hydroxymethyl) propane-1,3-diol hydrochloride was obtained after filtration.

7. The hexadecane tromethamine compound according to claim 6, which is characterized in that the concentration of the hydrochloric acid was 1 M.

8. The hexadecane tromethamine compound according to claim 1, which is characterized in that the hexadecyl trometamol salt was synthesized by the reaction of the hexadecyl tromethamine with acid.

9. The application of the hexadecane tromethamine compound according to any of claims 1-8 is in preparing anti-tumor drugs.

10. The application of the hexadecane tromethamine compound according to any of claims 1-8 is in preparing antifungal drugs.
